(19) United States
(12) Patent Application Publication
(10) Pub. No.: US 2020/0100889 A1
(43) Pub. Date: Apr. 2, 2020

(54) RADIOPACITY MODULATED RADIOPAQUE MARKER AND STENT GRAFT USING SAME

(71) Applicant: Cook Medical Technologies LLC, Bloomington, IN (US)

(72) Inventors: Davorin Skender, Bloomington, IN (US); Ralf Spindler, Solsberry, IN (US)

(21) Appl. No.: 16/149,820

(22) Filed: Oct. 2, 2018

(51) Int. Cl. A61F 2/07

(52) U.S. Cl. CPC A61F 2/07; A61F 2250/0098; A61F 2210/0014; A61F 2220/0075; A61F 2210/009; A61F 2210/0076

(57) ABSTRACT

In one aspect, a radiopaque marker for a medical implant, such as a stent graft for aortic aneurism repair, includes a first radiopaque component connected to a second radiopaque component by a connection. The first radiopaque component includes a non-biodegradable radiopaque body. The radiopaque marker has a first configuration and a second configuration that differ from one another in at least one of volume and shape such that the first and second configurations produce different radiographic images. This change in radiopacity can provide useful information to a surgeon in the event that reintervention becomes necessary.

### TECHNICAL FIELD

The present disclosure relates generally to radiopaque markers for medical implants, and more particularly to a radiopaque marker that changes to produce different radiographic images.

### BACKGROUND

Endovascular treatment of aortic aneurisms has a very high initial success rate for implants and clinical procedures. As the average age of the treated patient population becomes younger and the older population lives longer, the current generation of devices needs to last longer. Nevertheless, their can sometimes require secondary and even tertiary interventions. With these reinterventions, additional devices are often placed, including more stent grafts. The ability to accurately identify device land marks for proper placement can become confusing with radiopaque markers from the initial implant, when the secondary implant is co-located. Visualization on the fluoroscopy monitors can become crowded and confusing.

The present disclosure is directed toward one or more of the problems set forth above.

### SUMMARY

A radiopacity modulated radiopaque marker for a medical implant, such as a stent graft, includes a first radiopaque component connected to a second radiopaque component by a connection. The first radiopaque component includes a non-biodegradable radiopaque body. The radiopaque marker has a first configuration and a second configuration that differs from the first configuration in at least one of volume and shape such that the first and second configurations produce different radiographic images.

In another aspect, a stent graft includes a fabric tube attached to a stent. At least one radiopacity modulated radiopaque marker is attached to at least one of the stent and the fabric tube.

### BRIEF DESCRIPTION OF THE DRAWINGS

FIG. 1 is a perspective schematic view of a radiopacity modulated radiopaque marker according to the present disclosure;

FIG. 2 is a perspective view of the radiopaque marker of FIG. 1 after changing from a first configuration to a second configuration;

FIG. 3 is a perspective schematic view of a radiopacity modulated radiopaque marker according to a second embodiment of the present disclosure;

FIG. 4 is a perspective view of the radiopacity modulated radiopaque marker of FIG. 3 after changing from a first configuration to a second configuration;

FIG. 5 is a schematic view of a stent graft with attached radiopacity modulated radiopaque markers in a first configuration;

FIG. 6 is a schematic view of the stent graft of FIG. 5 after the radiopacity radiopaque markers have changed to a second configuration;

FIG. 7 is a schematic view of a stent graft with radiopacity modulated radiopaque markers according to different aspect of the present disclosure; and

FIG. 8 is a schematic view of a stent graft of FIG. 7 after the radiopacity modulated radiopaque markers have changed to a second configuration.

### DETAILED DESCRIPTION

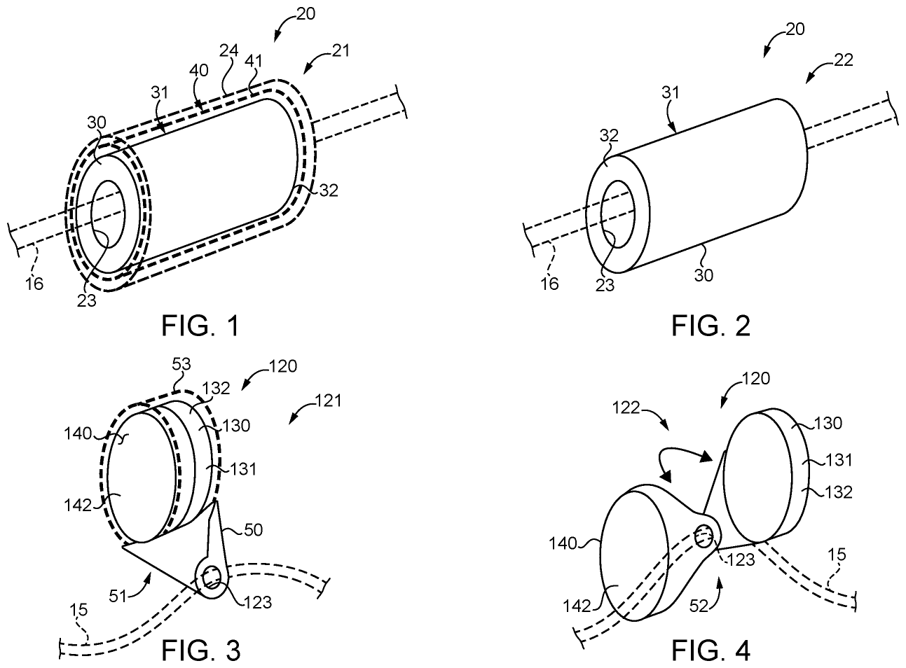

Referring initially to FIGS. 1 and 2, a radiopacity modulated radiopaque marker 20 for a medical implant includes a first radiopaque component 30 connected to a second radiopaque component 40 by a connection. In this example embodiment, the first radiopaque component 30 includes a non-biodegradable radiopaque body 31, which may comprise an integral block of metallic material 32 of the type well known in the art. Thus, first radiopaque component 30 may closely resemble known radiopaque markers both in form and material. In this example embodiment, the second radiopaque component includes a biodegradable coating 41 covering at least a portion of the exposed surface of first radiopaque component 30. Thus, in this example, a connection between the first radiopaque component 30 and the second radiopaque component 40 comprises the adherence contact between the biodegradable coating 41 and the integral block metallic material 32. The biodegradable coating 41 may be any suitable biodegradable coating known in the art, but may include a ferromanganese particles that function to produce a radiographic image in a manner well known in the art. Although not necessary, the biodegradable coating 41 and the underlying block of metallic material 32 may be protected prior to implantation by a suitable protective biodegradable coating 24, such as for instance polylactic acid (PLA). After implantation, the protective coating 24 will dissolve in the presence of blood. Thereafter, the underlying radiopaque biodegradable coating 41 will dissolve with the application of time and exposure to blood to eventually transform from a first configuration 21 as shown in FIG. 1 to a second configuration 22 as shown in FIG. 2. The first and second configurations 21, 22 differ from each other in at least one of volume and shape, such that the first and second configurations 21, 22 produce different radiographic images. In this instance, the shape of the radiopaque marker 20 remains substantially unchanged from the first configuration 21 to the second configuration 22, but the volume decreases and the loss of the biodegradable coating 41 could be expected to result in a radiographic image for the second configuration 22 of FIG. 2 being dimmer and slightly smaller than a radiographic image produced by the first configuration 21 of FIG. 1.

Those skilled in the art will appreciate that the radiopaque marker 20 can be attached to a medical implant, such as a stent, in any suitable manner including crimping, adhesive, sutures, or maybe even by utilizing a preformed hole 23 that receives a stent strut 16 as illustrated. Thus, any manner of connecting the radiopaque marker 22 a medical implant falls within the intended scope of the present disclosure. Those skilled in the art will appreciate that an alternative to the hole 23 defined by the block metallic material receiving a stent strut 16, hole 23 also can be sized to receive a suture to facilitate attachment to a medical implant, such as a stent graft or the like.

Referring now to FIGS. 3 and 4, a radiopacity modulated radiopaque marker 120 according to another embodiment of the present disclosure is shown in its first configuration 121 in FIG. 3 and its second configuration 122 in FIG. 4. This embodiment differs from the previous embodiment in that the first configuration 121 and the second configuration 122 differ from each other primarily in shape and to a lesser extent volume. Like the earlier embodiment, the radiopaque marker 120 includes a hole 123 therethrough to facilitate attachment to a medical implant, such as a stent graft, by way of a suture 15. Nevertheless, those skilled in the art will appreciate that the radiopaque marker 120 can be attached to an underlying medical implant using any known technique either alone or in combination, including but not limited to crimping, adhesives, sutures, and maybe even by receiving a stent strut through the hole 123 as discussed with regard to the previous embodiment. Radiopaque marker 120 includes a first radiopaque component 130 that includes a non-biodegradable radiopaque body 131 that may take the form of an integral block of metallic material 132, in the shape of a disk in this embodiment. The radiopaque marker 120 also includes a second radiopaque component 140 that may comprise a second integral block of metallic material 142 having a size and shape similar to the first radiopaque component 130. The two radiopaque components 130 and 140 are connected to one another by a connection 50 that changes shape responsive to a stimulus, such as time elapsed exposure to blood as in the previous embodiment. Connection 50 may be biased from a first shape 51 as shown in FIG. 3 toward a second shape 52 as shown in FIG. 4, which corresponds to the second configuration 122. This embodiment may utilize a restraint 53 that holds connection 50 against the bias until such time as the restraint 53 is released. Restraint 53 may comprise a biodegradable restraint that dissolves in the presence of blood to eventually release connection 50 to change from first shape 53 to second shape 52. Alternatively, restraint 53 may be triggered responsive to another alternative stimulus (other than blood or other body fluid) such as exposure to a magnetic field or exposure to light. For instance, restraint 53 may be released responsive to exposure to a magnetic field through the use of known ferromagnetic materials. Alternatively, the restraint 53 may be triggered for release responsive to exposure to light that may act on connection 50 by way of a shape memory polymer, such as BHECA, PCL, PCL(OH)₂ or [PLLA(OH)₂]. Still another stimulus that could be exploited to change a radiopaque marker from a first configuration to a second configuration as per the present disclosure could be heat and the use of shape memory materials, such as nitinol. Those skilled in the art will appreciate that the first configuration 121 and the second configuration 122 would produce substantially different radiographic images. This may be primarily due to the fact that the spatial relationship between the first integral block of radiopaque material 132 relative to the second integral block of radiopaque metallic material 142 is different in the first configuration 121 verses the second configuration 122.

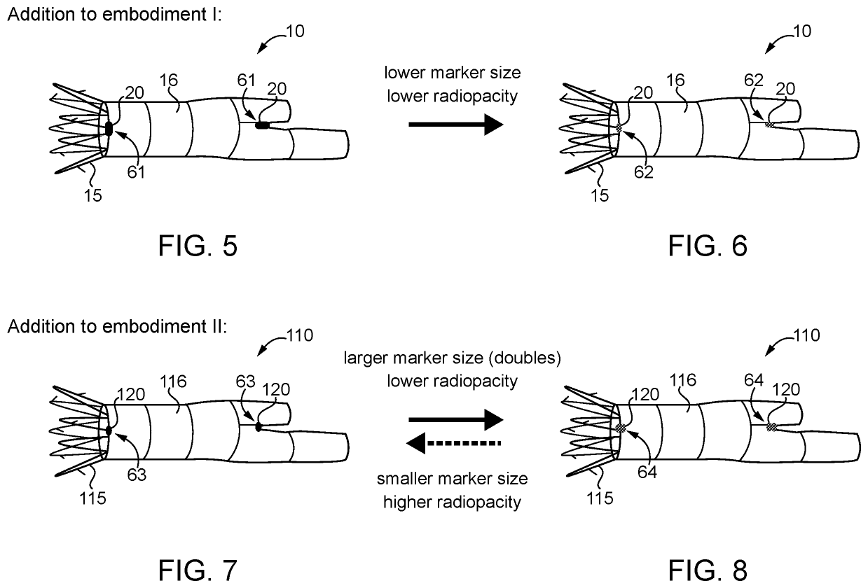

Referring now to FIGS. 5 and 6, a stent graft 10 includes a stent 15 with an attached fabric tube 16. Although the present disclosure could apply to virtually any medical implant, the present disclosure finds particular application to stent grafts, and more particularly to those associated with aortic aneurism repair as shown due to the higher likelihood of reintervention on the part of patients who have an aneurism in the aorta. Stent graft 10 includes two radiopacity modulated radiopaque markers 20 as described previously that present brighter radiopaque images 61 when the stent graft 10 is first implanted, but slightly smaller and dimmer radiopaque images 62 after some period of time and exposure to blood within the patient.

Referring now to FIGS. 7 and 8, a stent graft 110 includes a stent 115 and an attached fabric tube 116. Stent graft 110 also includes a pair of attached radiopacity modulated radiopaque markers 120 as described earlier after time and responsive to some stimulus such as exposure to blood, exposure to a magnetic field, exposure to light or maybe even heat, the radiopaque markers 120 transform from the smaller size presenting a smaller brighter radiopaque image with high radiopacity 63 toward a larger marker size with lower radiopacity radiographic image 64 as shown in FIG. 8. Those skilled in the art will appreciate that, alternatively, the first configuration may be shown by FIG. 8 and the second configuration may be shown by FIG. 7 such that the radiopaque markers may decrease in size and increase in radiopacity when transforming from the first configuration to the second configuration. Such might be the case if the markers were biased to the configuration shown in FIG. 3, but held initially in the configuration of FIG. 4 in a manner as previously described.

### INDUSTRIAL APPLICABILITY

The present disclosure finds general application in radiopaque markers for medical implants. The present disclosure finds specific application in usage with stent grafts. Finally, the present disclosure is particularly applicable to usage in stent grafts where there is a substantial possibility or likelihood that a patient might need reintervention, maybe years after the original stent graft is implanted in the patient. In such a case, the changed radiographic image presented by the radiopaque markers at the time of reintervention can be useful for the surgeon in differentiating a preexisting stent graft implanted maybe years ago from a medical implant during a reintervention procedure to avoid confusion with regard to positioning a later implanted stent graft during the reintervention. Those skilled in the art will appreciate that having the ability to easily differentiate between preexisting medical implants and new medical implants can provide the physician with useful information to produce better outcomes.

The following European style invention definitions are included to support multiple dependency type claims of the type favored in Europe and elsewhere. The definitions are as follows:

1. A radiopacity modulated radiopaque marker for a medical implant comprising: a first radiopaque component; a second radiopaque component connected to the first radiopaque component by a connection; wherein the first radiopaque component includes a non-biodegradable radiopaque body; wherein the radiopaque marker has first configuration and a second configuration that differs from the first configuration in at least one of volume and shape such that the first and second configurations produce different radiographic images.

2. The radiopaque marker of definition 1 wherein the non-biodegradable radiopaque body is an integral block of metallic material.

3. The radiopaque marker of any of definitions 1-2 wherein the second radiopaque component includes a biodegradable coating covering at least a portion of the integral block of metallic material.

4. The radiopaque marker of any of definitions 1-3 wherein at least one of the first radiopaque component, the second radiopaque component and the connection define a hole therethrough sized to receive at least one of a suture and a stent strut to facilitate attachment to a medical implant.

5. The radiopaque marker of any of definitions 1-4 wherein the integral block of metallic material is a first integral block of metallic material; the second radiopaque component includes a second integral block of metallic material; and the connection changes shape responsive to a stimulus.

6. The radiopaque marker of any of definitions 1-5 wherein the stimulus includes contact with blood.

7. The radiopaque marker of any of definitions 1-6 wherein the connection is biased from a first shape corresponding to the first configuration toward a second shape corresponding to the second configuration; a biodegradable restraint holding the connection in the first shape; and wherein the connection changes from the first shape to the second shape responsive to degradation of biodegradable restraint.

8. The radiopaque marker of any of definitions 1-7 wherein the stimulus includes exposure to a magnetic field.

9. The radiopaque marker of any of definitions 1-8 wherein the stimulus includes exposure to light.

10. The radiopaque marker of any of definitions 1-9 wherein a spatial relationship of the first integral block of radiopaque metallic material to the second integral block of radiopaque metallic material is different in the first configuration relative to the second configuration.

11. The radiopaque marker of any of definitions 1-10 wherein first configuration produces a brighter radiopaque image than the second configuration.

12. The radiopaque marker of any of definitions 1-11 wherein first configuration produces a smaller radiopaque image than the second configuration.

13. A stent graft comprising: a stent; a fabric tube attached to the stent; at least one radiopacity modulated radiopaque marker attached to at least one of the stent and the fabric tube; wherein the radiopaque marker includes a first radiopaque component and a second radiopaque component connected to the first radiopaque component by a connection; wherein the first radiopaque component includes a non-biodegradable radiopaque body; wherein the radiopaque marker has first configuration and a second configuration that differs from the first configuration in at least one of volume and shape such that the first and second configurations produce different radiographic images.

14. The stent graft of definition 13 wherein first configuration produces a brighter radiopaque image than the second configuration.

15. The stent graft of any of definitions 13-14 wherein first configuration produces a smaller radiopaque image than the second configuration.

16. The stent graft of any of definitions 13-15 wherein the non-biodegradable radiopaque body is an integral block of metallic material; and wherein the second radiopaque component includes a biodegradable coating in contact with the integral block of metallic material.

17. The stent graft of any definitons 13-16 wherein at least one of the first radiopaque component, the second radiopaque component and the connection define a hole therethrough; and the hole receives at least one of a suture tied to the fabric tube and a stent strut of the stent.

18. The stent graft of any of definitions 13-17 wherein the integral block of metallic material is a first integral block of metallic material; the second radiopaque component includes a second integral block of metallic material; and the connection changes shape responsive to a stimulus.

19. The stent graft of any of definitions 13-18 wherein the stimulus includes contact with blood.

20. The stent graft of any of definitions 13-19 wherein the stimulus includes exposure to at least one of a magnetic field and light.

The present description is for illustrative purposes only, and should not be construed to narrow the breadth of the present disclosure in any way. Thus, those skilled in the art will appreciate that various modification might be made to the presently disclosed embodiments without departing from the full and fair scope and spirit of the present disclosure. Other aspects, features and advantages will be apparent upon an examination of the attached drawings and appended claims.

What is claimed is:

1. A radiopacity modulated radiopaque marker for a medical implant comprising:
a first radiopaque component;
a second radiopaque component connected to the first radiopaque component by a connection;
wherein the first radiopaque component includes a non-biodegradable radiopaque body;
wherein the radiopaque marker has first configuration and a second configuration that differs from the first configuration in at least one of volume and shape such that the first and second configurations produce different radiographic images.

2. The radiopaque marker of claim 1 wherein the non-biodegradable radiopaque body is an integral block of metallic material.

3. The radiopaque marker of claim 2 wherein the second radiopaque component includes a biodegradable coating covering at least a portion of the integral block of metallic material.

4. The radiopaque marker of claim 1 wherein at least one of the first radiopaque component, the second radiopaque component and the connection define a hole therethrough sized to receive at least one of a suture and a stent strut to facilitate attachment to a medical implant.

5. The radiopaque marker of claim 1 wherein the integral block of metallic material is a first integral block of metallic material;
the second radiopaque component includes a second integral block of metallic material; and
the connection changes shape responsive to a stimulus.

6. The radiopaque marker of claim 5 wherein the stimulus includes contact with blood.

7. The radiopaque marker of claim 6 wherein the connection is biased from a first shape corresponding to the first configuration toward a second shape corresponding to the second configuration;
a biodegradable restraint holding the connection in the first shape; and
wherein the connection changes from the first shape to the second shape responsive to degradation of biodegradable restraint.

8. The radiopaque marker of claim 5 wherein the stimulus includes exposure to a magnetic field.

9. The radiopaque marker of claim 5 wherein the stimulus includes exposure to light.

10. The radiopaque marker of claim 5 wherein a spatial relationship of the first integral block of radiopaque metallic material to the second integral block of radiopaque metallic material is different in the first configuration relative to the second configuration.

11. The radiopaque marker of claim 1 wherein first configuration produces a brighter radiopaque image than the second configuration.

12. The radiopaque marker of claim 1 wherein first configuration produces a smaller radiopaque image than the second configuration.

13. A stent graft comprising:
a stent;
a fabric tube attached to the stent;
at least one radiopacity modulated radiopaque marker attached to at least one of the stent and the fabric tube;
wherein the radiopaque marker includes a first radiopaque component and a second radiopaque component connected to the first radiopaque component by a connection;
wherein the first radiopaque component includes a non-biodegradable radiopaque body;
wherein the radiopaque marker has first configuration and a second configuration that differs from the first configuration in at least one of volume and shape such that the first and second configurations produce different radiographic images.

14. The stent graft of claim 13 wherein first configuration produces a brighter radiopaque image than the second configuration.

15. The stent graft of claim 13 wherein first configuration produces a smaller radiopaque image than the second configuration.

16. The stent graft of claim 13 wherein the non-biodegradable radiopaque body is an integral block of metallic material; and
wherein the second radiopaque component includes a biodegradable coating in contact with the integral block of metallic material.

17. The stent graft of claim 13 wherein at least one of the first radiopaque component, the second radiopaque component and the connection define a hole therethrough; and
the hole receives at least one of a suture tied to the fabric tube and a stent strut of the stent.

18. The stent graft of claim 13 wherein the integral block of metallic material is a first integral block of metallic material;
the second radiopaque component includes a second integral block of metallic material; and
the connection changes shape responsive to a stimulus.

19. The stent graft of claim 18 wherein the stimulus includes contact with blood.

20. The stent graft of claim 5 wherein the stimulus includes exposure to at least one of a magnetic field and light.
